SEQUENCE LISTING (14 sequences)

SEQ ID NO: 1 - DNA, 20 bp, Homo sapiens
agcttatcatgcctggtcaa

SEQ ID NO: 2 - DNA, 20 bp, Homo sapiens
gggtacggagaagtcttgct

SEQ ID NO: 3 - DNA, 21 bp, Homo sapiens
aggttccgctatcgtgggcat

SEQ ID NO: 4 - DNA, 2082 bp, Homo sapiens
atcactgtagtagtagctggaaagagaaatctgtgactccaattagccagttcctgcagaccttgtgaggactagaggaagaatgctcctggctgttttgtactgcctgctgtggagtttccagacctccgctggccatttccctagagcctgtgtctcctctaagaacctgatggagaaggaatgctgtccaccgtggagcggggacaggagtccctgtggccagctttcaggcagaggttcctgtcagaatatccttctgtccaatgcaccacttgggcctcaatttcccttcacaggggtggatgaccgggagtcgtggccttccgtcttttataataggacctgccagtgctctggcaacttcatgggattcaactgtggaaactgcaagtttggcttttggggaccaaactgcacagagagacgactcttggtgagaagaaacatcttcgatttgagtgccccagagaaggacaaattttttgcctacctcactttagcaaagcataccatcagctcagactatgtcatccccatagggacctatggccaaatgaaaaatggatcaacacccatgtttaacgacatcaatatttatgacctctttgtctggatgcattattatgtgtcaatggatgcactgcttgggggatctgaaatctggagagacattgattttgcccatgaagcaccagcttttctgccttggcatagactcttcttgttgcggtgggaacaagaaatccagaagctgacaggagatgaaaacttcactattccatattgggactggcgggatgcagaaaagtgtgacatttgcacagatgagtacatgggaggtcagcaccccacaaatcctaacttactcagcccagcatcattcttctcctcttggcagattgtctgtagccgattggaggagtacaacagccatcagtctttatgcaatggaacgcccgagggacctttacggcgtaatcctggaaaccatgacaaatccagaaccccaaggctcccctcttcagctgatgtagaattttgcctgagtttgacccaatatgaatctggttccatggataaagctgccaatttcagctttagaaatacactggaaggatttgctagtccacttactgggatagcggatgcctctcaaagcagcatgcacaatgccttgcacatctatatgaatggaacaatgtcccaggtacagggatctgccaacgatcctatcttccttcttcaccatgcatttgttgacagtatttttgagcagtggctccgaaggcaccgtcctcttcaagaagtttatccagaagccaatgcacccattggacataaccgggaatcctacatggttccttttataccactgtacagaaatggtgatttctttatttcatccaaagatctgggctatgactatagctatctacaagattcagacccagactcttttcaagactacattaagtcctatttggaacaagcgagtcggatctggtcatggctccttggggcggcgatggtaggggccgtcctcactgccctgctggcagggcttgtgagcttgctgtgtcgtcacaagagaaagcagcttcctgaagaaaagcagccactcctcatggagaaagaggattaccacagcttgtatcagagccatttataaaaggcttaggcaatagagtagggccaaaaagcctgacctcactctaactcaaagtaatgtccaggttcccagagaatatctgctggtatttttctgtaaagaccatttgcaaaattgtaacctaatacaaagtgtagccttcttccaactcaggtagaacacacctgtctttgtcttgctgttttcactcagcccttttaacattttcccctaagcccatatgtctaaggaaaggatgctatttggtaatgaggaactgttatttgtatgtgaattaaagtgctcttattttaaaaaattgaaataattttgatttttgccttctgattatttaaagatctatatatgttttattggccccttctttattttaataaaacagtgagaaatctaaaaaaaaaaaaaaaaaa

SEQ ID NO: 5 - DNA, 16 bp, Homo sapiens
cgccagaagtgcggct

SEQ ID NO: 6 - DNA, 17 bp, Homo sapiens
cggcccgagagatacgc

SEQ ID NO: 7 - DNA, 13 bp, Homo sapiens
atccggcggccac

SEQ ID NO: 8 - DNA, 23 bp, Homo sapiens
actatggccttgtctgggatctc

SEQ ID NO: 9 - DNA, 25 bp, Homo sapiens
agatatggaacctgaagttgcactg

SEQ ID NO: 10 - DNA, 18 bp, Homo sapiens
caccatctcagccacagc

SEQ ID NO: 11 - DNA, 57 bp, Homo sapiens
cgccagaagtgcggctgggatccggcggccacctgcacctgcgtatctctcgggccg

SEQ ID NO: 12 - DNA, 89 bp, Homo sapiens
actatggccttgtctgggatctcagattttggcaacatctcagccacagcgggtgaaaactacagtgtcgtctcctgggtccccaagga

SEQ ID NO: 13 - DNA, 137 bp, Homo sapiens
agcttatcatgcctggtcaagaagcaggccttgggcaggttccgctgatcgtgggcatcttgctggtgttgatggctgtggtccttgcattatgaagcaagacttctccgtacccctctgatatataggcgcagact

SEQ ID NO: 14 - DNA, 70 bp, Homo sapiens
tctgctggtatttttctgtaaagaccatttgcaaaattgtaacctaatacaaagtgtagccttcttccaa

(19) United States
(12) Patent Application Publication
(10) Pub. No.: US 2011/0009288 A1
(43) Pub. Date: Jan. 13, 2011

(54) METHODS AND REAGENTS FOR THE EARLY DETECTION OF MELANOMA

(76) Inventors: Dmitri TALANTOV, San Diego, CA (US); John F. Palma, Carlsbad, CA (US); Tatiana Vener, Stirling, NJ (US); Tim Jatkoe, Gladstone, NJ (US); Haiying Wang, Bridgewater, NJ (US); Yixin Wang, San Diego, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/832,462

(22) Filed: Jul. 8, 2010

Related U.S. Application Data
(60) Provisional application No. 61/223,894, filed on Jul. 8, 2009.

(51) Int. Cl. C40B 30/04; C12Q 1/68

(52) U.S. Cl. 506/9; 435/6

(57) ABSTRACT

An assay for identifying early stage malignant melanocyte in biopsy tissues is provided by determining whether differential expression of a particular gene indicative of melanoma exceed a cut-off value.

### CROSS-REFERENCE TO RELATED APPLICATIONS

This application is a non-provisional of U.S. Provisional Patent Application No. 61/223,894, filed Jul. 8, 2009, which is hereby incorporated by reference in its entirety.

### BACKGROUND OF THE INVENTION

Cutaneous malignant melanoma is a serious health are problem with at least 62,000 new, invasive melanoma cases diagnosed in people expected in 2008 in the United States, among which 8,200 will die of this disease. The incidence of melanoma continues to increase faster than that of any other malignancy. Also, it is one of the most common cancers in young adults and, thus, is the number two type of cancer in terms of average years of life lost.

Certain proteins have been shown to be associated with melanoma and its metastases. These proteins or their activities have been used in immunohistochemistry to identify metastases and include L1CAM (Thies et al. (2002); Fogel et al. (2003)); and S-100 (Diego et al. (2003)). High-density microarrays have been applied to simultaneously monitor expression of thousands of genes in biological samples. Studies have resulted in the identification of genes differentially expressed in benign and malignant lesions, as well as genes that might be of prognostic value. Luo et al. (2001); and Wang et al. (2004). Gene expression profiling of malignant melanoma was performed with a microarray containing probes that monitor the expression of 8,150 mRNA transcripts. Bittner et al. (2000). These researchers identified several genes that might be associated with aggressive tumor behavior. In recent work, the comparison of gene expression profiles of melanoma and normal melanocyte cell lines led to the identification of differentially expressed genes and pathways modulated in melanoma. Takeuchi et al. (2004). Additionally, prostate differentiation factor GDF15, the adhesion molecule L1CAM, and kinesin-like 5, osteopontin, cathepsin B, cadherin 3, and presenilin 2 were identified as promising markers for melanoma detection. (Talantov et al. (2005); Wang et al. (2007)).

### SUMMARY OF THE INVENTION

The present invention provides a method of identifying a melanoma by: obtaining a tissue sample; assaying and measuring the expression levels in the sample for genes encoding mRNA corresponding to SILV (me20m) (SEQ. ID NO: 1-3), and tyorsinase (TYR) (SEQ. ID NO: 4). TYR was used as a mormalization control that confirms the presence of melanocytes in the tested sample. The invention further provides a method of identifying a melanoma by obtaining a tissue sample; and assaying and measuring the expression levels in the sample of genes encoding mRNA corresponding to one or both of GDF15 or L1CAM and recognized by the primer/probe sets SEQ. ID NOs.: 5-7 and 8-10, respectively, where gene expression is above a pre-determined cut-off is indicative of the presence of a melanoma.

The invention also provides a method of distinguishing a malignant melanocyte from a benign melanocyte by obtaining a tissue sample; and assaying and measuring the expression levels in the sample of genes encoding SILV where the gene expression levels above pre-determined cut-off is indicative of the presence of a melanoma in the sample.

The invention also provides a method of distinguishing a malignant melanocyte from a benign melanocyte by obtaining a tissue sample; and assaying and measuring the expression levels in the sample for genes encoding one or both of GDF15 and L1CAM and recognized by the primer/probe sets SEQ/ID NO.: 5-7 and 8-10. Gene expression levels above pre-determined cut-off is indicative of the presence of a melanoma in the sample.

The invention further provides a method of determining patient treatment protocol by obtaining a tissue sample from the patient; and assaying and measuring the expression levels in the sample of genes encoding SILV where the gene expression levels above pre-determined cut-off levels are indicative of the presence of a melanoma in the sample.

The invention further provides a method of determining patient treatment protocol by obtaining a tissue sample from the patient; and assaying and measuring the expression levels in the sample of genes encoding one or both of GDF15 and L1CAM recognized by the primer/probe sets SEQ. ID NOS.: 5-7 and 8-10 where the gene expression levels above pre-determined cut-off levels are indicative of the presence of a melanoma in the sample.

The final Marker is SILV and is defined herein as the gene encoding any variant, allele etc. SILV is also described as MELANOCYTE PROTEIN 17; PMEL17; PREMELANOSOMAL PROTEIN; PMEL; GP100; ME20; SI; SIL; D12S53E and represented by Accession No. NM_{—}006928.3. The invention further provides a kit for conducting an assay to determine the presence of melanoma in a cell sample comprising: nucleic acid amplification and detection reagents.

The invention further provides primer/probe sets for amplification and detection of PCR products obtained in the inventive methods. These sets include the following:

| SEQ. ID NO: 1, SILV, | |
|---|---|
| Forward primer, | AGCTTATCATGCCTGGTCAA |
| SEQ. ID NO: 2, SILV, | |
| Reverse primer, | GGGTACGGAGAAGTCTTGCT |
| SEQ. ID NO: 3, SILV, | |
| Probe, | FAM-AGGTTCCGCTATCGTGGGCAT-BHQ1 |
| SEQ. ID NO: 4, ABI AoD*, Hs00165976_m1 | |
| SEQ. ID NO: 5, GDF15, | |
| Forward primer, | CGCCAGAAGTGCGGCT |
| SEQ. ID NO: 6, GDF15, | |
| Reverse primer, | CGGCCCGAGAGATACGC |
| SEQ. ID NO: 7, GDF15, | |
| MGB Pobe, | FAM-ATCCGGCGGCCAC |
| SEQ. ID NO: 8, L1CAM, | |
| Forward primer, | ACTATGGCCTTGTCTGGGATCTC |
| SEQ. ID NO: 9, L1CAM, | |
| Reverse primer, | AGATATGGAACCTGAAGTTGCACTG |
| SEQ. ID NO: 10, L1CAM, | |
| MGB Pobe, | FAM-CACCATCTCAGCCACAGC |

The invention further provides amplicons obtained by PCR methods utilized in the inventive methods. These amplicons include the following:

SEQ. ID NO: 11, GDF15 PCR amplicon:
CGCCAGAAGTGCGGCTGGGATCCGGCGGCCACCTGCACCTGCGTATC
TCTCGGGCCG
SEQ. ID NO: 12 L1CAM PCR amplicon:
ACTATGGCCTTGTCTGGGATCTCAGATTTTGGCAACATCTCAGCCACA
GCGGGTGAAAACTACAGTGTCGTCTCCTGGGTCCCCAAGGA
SEQ. ID NO: 13 SILV PCR amplicon:
AGCTTATCATGCCTGGTCAAGAAGCAGGCCTTGGGCAGGTTCCGCTGA
TCGTGGGCATCTTGCTGGTGTTGATGGCTGTGGTCCTTGCATTATGAA
GCAAGACTTCTCCGTACCCCTCTGATATATAGGCGCAGACT
SEQ. ID NO: 14 TYR PCR amplicon:
TCTGCTGGTATTTTTCTGTAAAGACCATTTGCAAAATTGTAACCTAAT
ACAAAGTGTAGCCTTCTTCCAA

### BRIEF DESCRIPTION OF THE DRAWINGS

FIG. 1 is a graph showing the performance of SILV, GDF15 and L1CAM in distinguishing benign and malignant skin lesions.

FIG. 2 is a graph plotting sensitivity versus specificity of SILV, GDF15 and L1CAM in distinguishing unequivocal melanomas and benign nevi.

FIG. 3 is a graph of SILV performance in distinguishing between melanoma and atypical nevi.

FIG. 4 is a graph plotting sensitivity versus specificity of SILV in distinguishing unequivocal melanomas and benign nevi.

### DETAILED DESCRIPTION OF THE INVENTION

The present invention provides methods of qualitatively and quantitatively identifying a melanoma; distinguishing a malignant melanocyte from a benign melanocyte; diagnosing melanocytic lesions with uncertain pathological features; and determining a melanoma patient treatment protocol. The methods further provide aids in patient prognosis, patient monitoring and drug development. The methods rely on assaying and measuring expression levels of SILV (me20m) as a final Marker for melanoma biopsy assays where a certain level of gene expression, relative to TYR normalization control, is indicative of the presence of a malignant melanocyte in the sample assayed.

The present invention focuses on the utility of identified gene expression markers to diagnose malignant melanoma among various skin lesions, including lesions with uncertain morphological features or suspicious primary melanocytic lesions termed equivocal cases using paraffin-embedded (“FFPE”) tissues. This setting is where discrepancies between the opinions of dermatopathologists occur. Thus, the utility of this invention is to identify gene expression markers that differentiate benign melanocytic skin lesions from primary melanomas in gene expression assays, based on RT-PCR, for the diagnosis of melanoma in suspicious lesions.

Total RNA was isolated from 47 primary melanoma, 48 benign skin nevi, and 98 atypical/suspicious nevi including 48 atypical nevi and 50 melanomas (as assigned by dermatopathologists) tissue specimens were analyzed using RT-PCR. Differential gene expression of three melanoma specific genes, SILV, GDF15, and L1CAM, were tested by a one-step quantitative RT-PCR assay on melanoma, benign nevi and atypical/suspicious skin samples. The results demonstrated the ability of using SILV as a final Marker to differentiate clinically relevant tissue samples containing benign or malignant melanocytes.

High-density cDNA and oligonucleotide microarrays allow simultaneous monitoring of the expression of thousands of genes. Microarray technology provides a quantitative measurement of mRNA abundance and has gained acceptance as a tool for marker discovery based on gene expression. In the context of cancer research, microarray analysis has identified genes differentially expressed in benign and malignant lesions for different cancer types or that have prognostic significance. Luo et al. (2001); Su et al. (2001); Henshall et al. (2003); and Wang et al. (2004). The first gene expression profiling of malignant melanoma used a microarray containing probes for 8,150 mDNA transcripts and identified genes that might be associated with aggressive tumor behavior. Bittner et al. (2000). Since the samples analyzed in their study did not include tissues containing normal or benign melanocytes, differentially expressed genes in malignant melanoma were not identified. In contrast to normal skin, melanocyte content in benign nevi is close to that in melanoma.

In another study, two pooled samples derived from either melanoma or benign nevi tissues were hybridized to a cDNA array and genes preferentially expressed in melanoma- or nevi-derived samples were found. Seykora et al. (2003). Other researchers used subtractive hybridization or analysis of SAGE libraries generated on melanoma cell lines, for monitoring gene expression in melanoma. Hipfel et al. (2000); and Weeraratna (2004). Recently, the comparison of gene expression profiles of a few melanoma and melanocyte cell lines led to the identification of differentially expressed genes and pathways modulated in melanoma. Hoek et al. (2004). While these studies provide a solid foundation for melanoma genomics, there is no marker that can clearly differentiate melanoma from benign tissue. Several markers currently used such as tyrosinase, HMB-45, mart-a/Melanin-a and MITF have not proven to have prognostic value for melanocytic tumor identification Phsie et al. (2008) Consequently, these markers have found limited clinical use.

As disclosed in United States Patent Publication No. 20070154889, incorporated herein in its entirety by reference, PCR was demonstrated to be have sufficient specificity and sensitivity to detect metastasis of melanoma. In the present invention, a method with improved diagnostic performance in differentiating melanoma from non-melanoma lesions from primary skin biopsies is provided. The assay of the invention is useful in diagnosing clear-cut, or unequivocal, from suspicious, or equivocal, lesions. Thus, the instant invention may find particular utility in testing of early stage tissue samples. Preferably, a probability measurement will distinguish tissues having melanoma relative to benign melanocyte or normal tissue.

The methods of the invention employ a rapid technique for extracting nucleic acids from FFPE tissue samples and a method of amplifying and detecting nucleic acid fragments indicative of melanoma in primary skin lesions. The nucleic acid fragments qualitatively and quantitatively measure mRNA encoded by the Marker gene. Tissue samples include skin lesions derived from punch, needle, excisional or shave biopsies. The methods provided herein allow for melanoma detection in primary skin biopsy samples allowing a physician to determine whether to immediately implement an appropriate treatment protocol for early stage disease. In the methods of the invention, it is important to adequately sample the tissue used to conduct the assay. This includes proper excision and processing of the tissue sample as well as extraction of RNA. Once obtained, it is important to process the tissue samples properly so that any cancerous cells present are detected.

In one method of the invention, RNA is isolated from an FFPE tissue sample block. Any suitable commercially available paraffin kit may be used, such as High Pure RNA Paraffin Kit from Roche (Cat. #3270289). Preferably, the FFPE samples are sectioned according to the size of the embedded tumor as follows: ≦2 to 5 mm sectioned to 9×10 μm; ≧6 to 8 mm sectioned to 6×10 μm. Sections are de-paraffinized according to the manufacturer's instructions and the isolated RNA may be stored in RNase-free water at −80° C.

In the case of measuring mRNA levels to determine gene expression, assays can be by any means known in the art and include methods such as PCR, Rolling Circle Amplification (RCA), Ligase Chain Reaction (LCR), Strand Displacement Amplification (SDA), Nucleic Acid Sequence Based Amplification (NASBA), and others. The rapid molecular diagnostics involved are most preferably quantitative PCR methods, including QRT-PCR. Detection can be by any method known in the art including microarrays, gene chips and fluorescence.

A typical PCR includes multiple amplification steps, or cycles that selectively amplify target nucleic acid species. A typical PCR includes three steps: a denaturing step in which a target nucleic acid is denatured; an annealing step in which a set of PCR primers (forward and backward primers) anneal to complementary DNA strands; and an elongation step in which a thermostable DNA polymerase elongates the primers. By repeating this step multiple times, a DNA fragment is amplified to produce an amplicon, corresponding to the target DNA sequence. Typical PCR includes 20 or more cycles of denaturation, annealing and elongation. Often, the annealing and elongation steps can be performed concurrently, in which case the cycle contains only two steps.

In one embodiment of the invention, the RT-PCR amplification reaction may be conducted in a time so that the lengths of each of these steps can be in the seconds range, rather than minutes. Specifically, with certain new thermal cyclers being capable of generating a thermal ramp rate of at least about 5C.° per second, RT-PCR amplifications in 30 minutes or less are used. More preferably, amplifications are conducted in less than 25 minutes. With this in mind, the following times provided for each step of the PCR cycle do not include ramp times. The denaturation step may be conducted for times of 10 seconds or less. In fact, some thermal cyclers have settings for “0 seconds” which may be the optimal duration of the denaturation step. That is, it is enough that the thermal cycler reaches the denaturation temperature. The annealing and elongation steps are most preferably less than 10 seconds each, and when conducted at the same temperature, the combination annealing/elongation step may be less than 10 seconds. Some homogeneous probe detection methods, may require a separate step for elongation to maximize rapid assay performance. In order to minimize both the total amplification time and the formation of non-specific side reactions, annealing temperatures are typically above 50° C. More preferably annealing temperatures are above 55° C.

A single combined reaction for RT-PCR, with no experimenter intervention, is desirable for several reasons: (1) decreased risk of experimenter error; (2) decreased risk of target or product contamination; and (3) increased assay speed. The reaction can consist of either one or two polymerases. In the case of two polymerases, one of these enzymes is typically an RNA-based DNA polymerase (reverse transcriptase) and one is a thermostable DNA-based DNA polymerase. To maximize assay performance, it is preferable to employ a form of “hot start” technology for both of these enzymatic functions. U.S. Pat. Nos. 5,411,876 and 5,985,619 provide examples of different “hot start” approaches and incorporated herein by reference. Preferred methods include the use of one or more thermoactivation methods that sequester one or more of the components required for efficient DNA polymerization. U.S. Pat. Nos. 5,550,044 and 5,413,924 describe methods for preparing reagents for use in such methods and are incorporated herein by reference. U.S. Pat. No. 6,403,341 describes a sequestering approach that involves chemical alteration of one of the PCR reagent components and is incorporated herein by reference. In the most preferred embodiment, both RNA- and DNA-dependent polymerase activities reside in a single enzyme. Other components that are required for efficient amplification include nucleoside triphosphates, divalent salts and buffer components. In some instances, non-specific nucleic acid and enzyme stabilizers may be beneficial.

In the preferred RT-PCR, the amounts of certain reverse transcriptase and the PCR components are atypical in order to take advantage of the faster ramp times of some thermal cyclers. Specifically, the primer concentrations are very high.

Typical gene-specific primer concentrations for reverse transcriptase reactions are less than about 20 nM. To achieve a rapid reverse transcriptase reaction on the order of one to two minutes, the reverse transcriptase primer concentration is raised to greater than 20 nM, preferably at least about 50 nM, and typically about 100 nM. Standard PCR primer concentrations range from 100 nM to 300 nM. Higher concentrations may be used in standard PCR to compensate for Tm variations. However, for the purposes herein, the referenced primer concentrations are for circumstances where no Tm compensation is needed. Proportionately higher concentrations of primers may be empirically determined and used if Tm compensation is necessary or desired. To achieve rapid PCR, the PCR primer concentrations typically are greater than 250 nM, preferably greater than about 300 nM and typically about 500 nM.

Commercially used diagnostics also preferably employ one or more internal positive control that confirms the operation of a particular amplification reaction in case of a negative result. Potential causes of false negative results that must be controlled in an RT-PCR include: inadequate RNA quantity, degradation of RNA, inhibition of RT and/or PCR and experimenter error.

In the case of measuring protein levels to determine gene expression, any method known in the art is suitable provided it results in adequate specificity and sensitivity. For example, protein levels can be measured by binding to an antibody or antibody fragment specific for the protein and measuring the amount of antibody-bound protein. Antibodies can be labeled by radioactive, fluorescent or other detectable reagents to facilitate detection. Methods of detection include, without limitation, enzyme-linked immunosorbent assay (ELISA) and immunoblot techniques.

The invention provides specificity and sensitivity sufficient to identify a malignant melanocyte in a tissue sample. The methods determine expression of a particular Marker gene, SILV, by measuring mRNA encoded by the Marker. The results presented herein show that SILV is a leading marker demonstrating clear discrimination between melanoma and benign, unequivocal cases as well as between different atypia subgroups in suspicious tissue group samples. The Marker SILV is defined herein as the gene encoding any variant, allele etc. including SEQ ID NO: 1-3.

In the methods of the invention, tyrosinase is used as a control gene to demonstrate the presence of melanocytes in the tissue sample and to normalize for melanocytic content. Tyrosinase is described by Mandelcorn-Monson et al. (2003); and U.S. Pat. No. 6,153,388 and is represented by Accession No. NM_{—}000372. Tyrosinase is also defined as the gene encoding mRNA recognized by the ABI assay on demand (Hs00165976_m1) with PCR amplicon SEQ ID NO: 14.

The specificity of any given amplification-based molecular diagnostic relies heavily, but not exclusively, on the identity of the primer sets. The primer sets are pairs of forward and reverse oligonucleotide primers that anneal to a target DNA sequence to permit amplification of the target sequence, thereby producing a target sequence-specific amplicon. The primers must be capable of amplifying Markers of the disease state of interest. In the case of the instant invention, the Marker is directed to melanoma.

The reaction must also contain some means of detection of a specific signal. This is preferably accomplished through the use of a reagent that detects a region of DNA sequence derived from polymerization of the target sequence of interest. Preferred reagents for detection give a measurable signal differential when bound to a specific nucleic acid sequence of interest. Often, these methods involve nucleic acid probes that give increased fluorescence when bound to the sequence of interest. Typically, the progress of the reactions of the inventive methods are monitored by analyzing the relative rates of amplicon production for each PCR primer set.

The invention further includes primer/probe sets and their use in the claimed methods. The sequences IDs are: SEQ, ID Nos.: 1-10. Monitoring amplicon production may be achieved by a number of detection reagents and methods, including without limitation, fluorescent primers, and fluorogenic probes and fluorescent dyes that bind double-stranded DNA. Molecular beacons, Scorpions, and other detection schemes may also be used. A common method of monitoring a PCR employs a fluorescent hydrolysis probe assay. This method exploits the 5′ nuclease activity of certain thermostable DNA polymerases (such as Taq or Tfl DNA polymerases) to cleave an oligomeric probe during the PCR process.

The invention further provides amplicons obtained by PCR methods utilized in the inventive methods. These amplicons include the sequences: SEQ. ID Nos: 11-14.

The oligomer is selected to anneal to the amplified target sequence under elongation conditions. The probe typically has a fluorescent reporter on its 5′ end and a fluorescent quencher of the reporter at the 3′ end. So long as the oligomer is intact, the fluorescent signal from the reporter is quenched. However, when the oligomer is digested during the elongation process, the fluorescent reporter is no longer in proximity to the quencher. The relative accumulation of free fluorescent reporter for a given amplicon may be compared to the accumulation of the same amplicons for a control sample and/or to that of a control gene, such as, without limitation, β-Actin or PBGD to determine the relative abundance of a given cDNA product of a given RNA in a RNA population. Products and reagents for the fluorescent hydrolysis probe assay are readily available commercially, for instance from Applied Biosystems.

The most preferred detection reagents are TaqMan® probes (Roche Diagnostics, Branchburg, N.J.) and they are described in U.S. Pat. Nos. 5,210,015; 5,487,972; and 5,804,375 incorporated herein by reference. Essentially, these probes involve nucleic acid detection by virtue of the separation of a fluor-quencher combination on a probe through the 5′-3′ exonuclease activity of the polymerase used in the PCR. Any suitable fluorophore can be used for any of the Markers or controls. Such fluorophores include, without limitation, Texas Red, Cal Red, Fam, Cy3 and Cy5. In one embodiment, the following fluorophores correspond to the noted Markers: PLAB: Fam; L1CAM: Texas Red or Cal Red, tyrosinase: C1; PBGD: Cy5. Equipment and software also are readily available for controlling and monitoring amplicon accumulation in PCR and QRT-PCR including the Smart Cycler thermocylcer commercially available from Cepheid of Sunnyvale, Calif., and the ABI Prism 7900 Sequence Detection System, commercially available from Applied Biosystems.

In the commercialization of the described methods for QRT-PCR certain kits for detection of specific nucleic acids are particularly useful. In one embodiment, the kit includes reagents for amplifying and detecting Markers. Optionally, the kit includes sample preparation reagents and or articles (e.g., tubes) to extract nucleic acids from lymph node tissue. The kits may also include articles to minimize the risk of sample contamination (e.g., disposable scalpel and surface for lymph node dissection and preparation).

In a preferred kit, reagents necessary for the one-tube QRT-PCR process described above are included such as reverse transcriptase, a reverse transcriptase primer, a corresponding PCR primer set (preferably for Markers and controls), a thermostable DNA polymerase, such as Taq polymerase, and a suitable detection reagent(s), such as, without limitation, a scorpion probe, a probe for a fluorescent hydrolysis probe assay, a molecular beacon probe, a single dye primer or a fluorescent dye specific to double-stranded DNA, such as ethidium bromide. The primers are preferably in quantities that yield the high concentrations described above. Thermostable DNA polymerases are commonly and commercially available from a variety of manufacturers. Additional materials in the kit may include: suitable reaction tubes or vials, a barrier composition, typically a wax bead, optionally including magnesium; reaction mixtures (typically 10×) for the reverse transcriptase and the PCR stages, including necessary buffers and reagents such as dNTPs; nuclease- or RNase-free water; RNase inhibitor; control nucleic acid(s) and/or any additional buffers, compounds, co-factors, ionic constituents, proteins and enzymes, polymers, and the like that may be used in reverse transcriptase and/or PCR stages of QRT-PCR. Optionally, the kits include nucleic acid extraction reagents and materials. Instructions are also preferably included in the kits.

The following examples are provided to illustrate but not limit the claimed invention.

### Example 1

### Patient Clinical and Pathological Characteristics

204 FFPE skin biopsy tissue specimens were selected from patients with primary melanocytic skin lesions diagnosed at Georgetown University Hospital. The patient series included specimens with 102 unequivocal features of invasive melanoma or benign nevi and 102 specimens with various degrees of cellular atypia. These atypical specimens were initially classified as suspicious/atypical and subsequently resolved by expert dermatopathologists as atypical nevi or malignant melanoma. Two patient samples were excluded because of insufficient RNA yield (less than 350 ng) after a sample preparation step. An additional nine RNA samples were excluded due to the failure of the PCR control. The final sample set eligible for analysis consisted of 193 biopsy tissues (95% of the original sample set) representing 47 melanomas, 48 benign nevi and 98 atypical/suspicious, including 48 atypical nevi and 50 melanomas as assigned by dermatopathologists. A summary of the pathological and clinical characteristics of the melanoma samples is shown in Table 1.

TABLE 1

| | Advanced | | Severe | Moderate | Benign |
|---|---|---|---|---|---|
| Patient Characteristics | Melanoma | Melanoma | Atypia | Atypia | Nevi |
| Mean Age | 59 | 51 | 42 | 440 | 42 |
| Gender | | | | | |
| Female | 8 | 35 | 20 | 8 | 33 |
| Male | 6 | 48 | 15 | 5 | 15 |
| T Stage (thickness) | | | | | |
| Tis | | 21 | | | |
| T1 (<1 mm) | 1 | 62 | | | |
| T2 (1.01-2 mm) | 7 | | | | |
| T3 (2.01-4 mm) | 3 | | | | |
| T4 (>4 mm) | 1 | | | | |
| M | 2 | | | | |
| Diagnosis | | | | | |
| Superficial spreading melanoma | 6 | 50 | | | |
| Nodular melanoma | 5 | | | | |
| Melanoma in-situ | | 21 | | | |
| Lentigo maligna | | 11 | | | |
| Melanoma other | 3 | 1 | | | |
| Compound nevus | | | | | 31 |
| Inflamed compound nevus | | | | | 13 |
| Intradermal nevus | | | | | 4 |
| Atypical nevus severe atypia | | | 2 | | |
| Compound nevus severe atypia | | | 29 | | |
| Compound nevus moderate atypia | | | | 13 | |
| Junctional nevus severe atypia | | | 4 | | |
| Total n per category | 14 | 83 | 35 | 13 | 48 |
| Unequivocal melanoma, n = 47 | 12 | 35 | | | |
| Atypical melanoma, n = 50 | 2 | 48 | | | |
| Unequivocal benign, n = 48 | | | | | 48 |
| Atypical nevi, n = 48 | | | 35 | 13 | |

Samples were ordered from most benign to most malignant cases, based on the provided clinical data by pathology. Using a histological diagnosis, five major categories were created: advanced melanoma, melanoma, severe atypia, moderate atypia and benign nevi, with each of the 193 samples fitting into one of these groups. Lentigo maligna melanoma, melanoma in situ, and superficial invasive melanomas were combined into a single melanoma category. Two sets of melanomas with advanced features (superficial spreading with T2 and greater and nodular or metastatic) were added into an advanced melanoma group. A binary classification was based on splitting advanced melanomas and melanomas into malignant, and the remaining classes as benign. This stratification contained 97 malignant cases with 47 unequivocal and 50 severely atypical lesions classified as melanomas, and 96 benign cases representing 48 benign and 48 atypical nevi. All further data analysis described is presented for unequivocal cases classified into one of the 3 groups: advanced melanoma, melanoma and benign and for equivocal cases classified into one of the 4 groups: advanced melanoma, melanoma, severe and moderate atypia.

### Example 2

### Tissue Preparation

Two hundred four tissue samples were collected from individuals diagnosed with primary melanocytic skin lesions. All samples were collected using excisional, punch, or shave biopsy depending on lesion size, depth, and physician judgment and embedded in FFPE blocks.

Total RNA was isolated from FFPE blocks using a standard High Pure RNA paraffin Kit from Roche (catalogue # 3270289) with the following modifications. Paraffin embedded tissue samples were sectioned according to the size of the embedded tumor (2-5 mm or smaller=9×10 μm, 6-8 mm or greater=6×10 μm). Sections were de-paraffinized according to the manufacturer's instructions. The isolated RNA was stored in RNase free water at −80° C. until used.

The distribution by biopsy type and extracted RNA yield are presented in Tables 2a and 2b, respectively. Median RNA yields, corresponding to a total average of 10.5, 4.5 and 6 slides were equivalent among all three types of biopsies. No bias in assay performance was observed based on the differences in biopsy techniques.

TABLE 2A

| Pathology | | | | |
|---|---|---|---|---|
| Diagnosis | Excision | Punch | Shave | Total |
| Melanoma | 21 | 7 | 23 | 51 |
| Benign | 6 | 4 | 41 | 51 |
| Atypical/Suspicious | 39 | 22 | 41 | 102 |
| Total | 66 (32%) | 33 (16%) | 105 (52%) | 204 (100%) |

TABLE 2b

| | | | | Median | |
|---|---|---|---|---|---|
| | | | | RNA | |
| Biopsy | Number | Median | | Yield | Range |
| Type | (%) | Size | Section # | (ng) | (ng) |
| Excision | 66 (32) | 13 × 10.5 × 8 | 3-6 | 1360.6 | 496-26879 |
| Punch | 33 (16) | 8 × 7 × 3 | 6 | 1534.1 | 397-7368 |
| Shave | 105 (52) | 7 × 6 × 1.5 | 9-12 | 1400.4 | 318-12140 |

### Example 3

### Single One-Step qRTPCR Assays Using RNA-Specific Primers and Cutoff Establishment

Evaluation of expression of selected genes was carried out with one-step RT-PCR with RNA from melanoma, benign nevi, and atypical/suspicious FFPE tissue. The specimens included two series of samples: 1) unequivocal, or clear-cut, melanoma and benign nevi cases and 2.) samples with various degrees of atypia. Tyrosinase (“TYR”) was used as a housekeeping gene to control for the input quantity and quality of RNA in the reactions. DNase treatment was not used. Instead, primers or probes were designed to span an intron so they would not report on genomic DNA. All primer/probe sets were pre-screened on a set of 20 total RNA specimens isolated from 10 melanoma and 10 benign nevi FFPE tissues from a commercial vendor (Oncomatrix). The best performing primer-probe set was selected for each of the four markers. The sequences are listed in Table 3 below.

The gene expression markers GDF-15, SILV, and L1CAM along with the normalization control tyrosinase (“TYR”) were tested in the melanoma biopsy assay using a single reaction RT-PCR format on the ABI7900 platform. Single, one step qRT-PCR assays were run in accordance with the following protocol. 50 ng of total RNA was used for qRT-PCR. The total RNA was reverse transcribed using 40× Multiscribe and RNase inhibitor mix contained in the TAQMAN® One Step PCR Master Mix Reagents Kit (Applied Biosystems, Foster City, Calif.). The cDNA was then subjected to the 2× Master Mix using UNG and PCR amplification was carried out on the ABI 7900 HT Sequence Detection System (Applied Biosystems, Foster City, Calif.) in the 384-well format using a 10 μl reaction size. Each reaction was composed of 5.0 μl of 2× One Step RT-PCR Master Mix, 0.5 μl of primer/probe mix, 0.25 μl of 40× Multiscribe enzyme, and RNase Inhibitor Mix, 0.25 μl of dNTP and 4 μl of 12.5 ng/μl total RNA. The final primer/probe mix was composed of a final concentration of 900 nM of forward and reverse primers, listed in Table 3, and 250 nM of fluorescent probe. The dNTP mix contained a final concentration of 20 mM each of dATP, dGTP, dCTP, and dTTP. The reaction mixture was incubated at 48° C. for 30 min. for the reverse transcription, followed by an Amplitaq activation step of 95° C. for 10 minutes, and finally 40 cycles of 95° C. for 15 sec. denaturing and 60° C. for 1 minute anneal and extension. Sequences used in the reactions were as follows, each written in the 5′ to 3′ direction.

TABLE 3

| | Primer/ | | ID. |
|---|---|---|---|
| Symbol | Probe | Sequence | No. |
| GDF15 | Forward | CGCCAGAAGTGCGGCT | 5 |
| | Reverse | CGGCCCGAGGATACGC | 6 |
| | MGB Probe | FAM-ATCCGGCGGCCAC | 7 |
| L1CAM | Forward | ACTATGGCCTTGTCTGGGATCTC | 8 |
| | Reverse | AGATATGGAACCTGAAGTTGCACTG | 9 |
| | MGB probe | FAM-CACCATCTCAGCCACAGC | 10 |
| SILV | Forward | AGCTTATCATGCCTGGTCAA | 1 |
| | Reverse | GGGTACGGAGAAGTCTTGCT | 2 |
| | Probe | FAM-AGGTTCCGCTATCGTGGGCAT-BHQ1 | 3 |
| TYR | ABI AoD* | Hs00165976_m1 | 4 |

For each sample ΔCt=Ct (Target Gene)−Ct TYR was calculated. ΔCt has been widely used in clinical RT-PCR assays and was chosen as a straightforward method. Cronin et al. (2004).

The Ct values obtained from ABI7900 output files were used for data analysis. In the single reaction assay configuration, only samples generating TYR Ct<30 were analyzed, The Ct values for each of the markers are presented as raw Cts normalized against the melanocyte-specific marker, TYR, using the following equation:

Ct(normalized)=Ct(marker)−CT(TYR)

Diagnosis rendered by assay was compared with dermatopathological examination. To estimate assay performance, AUC values were calculated based on ROC curve analysis using R software package version 2.5.0. (team RDC www.r-project.org).

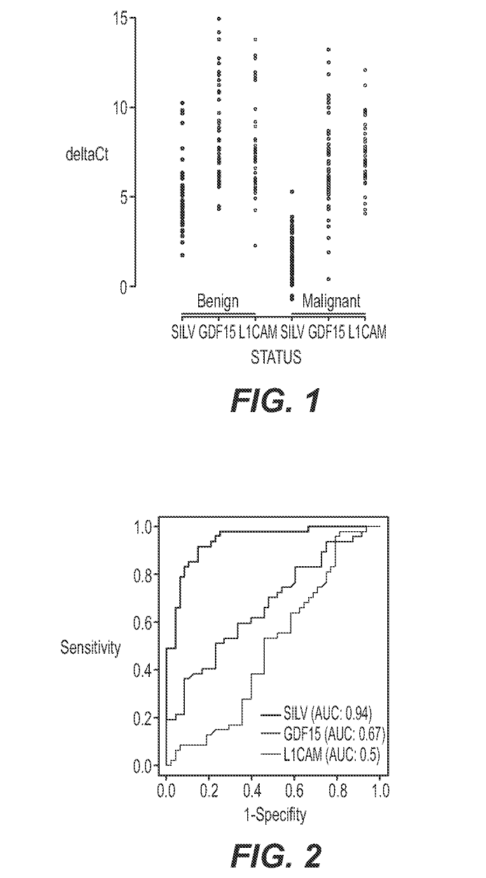

For clear-cut (unequivocal) cases, increased expression was demonstrated in melanoma compared to benign lesions for the three melanoma-specific markers (Table 4, FIG. 1a). Significant differential expression was observed for SILV and GDF15 between benign nevi and melanoma samples in clear-cut (unequivocal) cases (2.8- and 1.2-fold with p-values <0.001 and 0.003, respectively). However, L1CAM demonstrated much less differentiation with a fold change of 0.2 and no statistical significance for the difference (p=0.47) between benign and malignant clear-cut cases (FIG. 1). Thus, this marker was excluded from further analysis. SILV demonstrated the best performance with a linear response across the three patient groups (advanced melanoma, melanoma, and benign cases), representing continuously changing degrees of disease status as defined by pathology.

TABLE 4

| Marker | | | | | AUC |
|---|---|---|---|---|---|
| Normal- | | | | P-values | (classification |
| ized | Advanced | | Benign | (benign v | as benign or |
| to TYR | Melanoma | Melanoma | Nevi | malignant) | malignant) |
| L1CAM | 6.85 | 7.5 | 7.66 | 0.47 | 0.49 |
| SILV | 1.18 | 2.09 | 4.93 | <0.001 | 0.94 |
| GDF15 | 5.02 | 7.17 | 8.34 | 0.003 | 0.67 |

The performance of SILV and GDF15 was assessed for differentiation between unequivocal melanomas and benign nevi using a univariate ROC curve analysis. As shown in FIG. 2, AUC values were 0.94 and 0.67, respectively. Based on multivariate analysis with a linear regression model, the combination of SILV and GDF15 did not improve assay performance beyond the AUV of 0.94 in unequivocal cases. Therefore, GDF15 was not pursued further for analysis of suspicious (equivocal cases). Finally, normalization to TYR improved performance of SILV to 0.94 compared to 0.78 when using raw Ct values.

The performance of SILV was assessed further by comparing suspicious cases to unequivocal benign cases. The average ΔCt of SILV in the equivocal samples in each by histology, excluding advanced melanoma since n=2, was compared to the average ΔCt of the unequivocal benign group. The average ΔCt values and p-values for t-test comparisons to the unequivocal begin samples are listed in Table 5 below. SILV was significantly different between suspicious melanoma and each suspicious atypical group: melanoma versus severe atypia with a p-value=0.0077 and melanoma vs. moderate atypia with a p-value=0.0009.

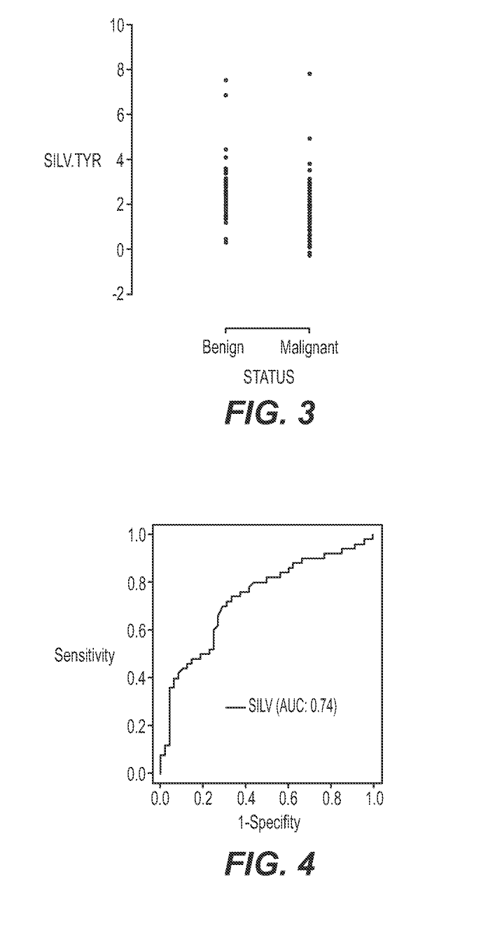

FIGS. 1 through 4 confirm that SILV is the leading marker and demonstrated clear discrimination between melanoma and benign equivocal cases as well as between different atypia subgroups in the suspicious group of tissue samples.

TABLE 5

| Marker | Normalized ΔCt Values | | | | P-Values | | |
|---|---|---|---|---|---|---|---|
| Normalized | | Severe | Moderate | Benign | Benign v. | Benign v. | Benign v. |
| to TYR | Melanoma | Atypia | Atypia | Unequivocal | Moderate | Severe | Melanoma |
| SILV | 1.7 | 2.49 | 3.15 | 4.93 | 0.002 | 3.35E−09 | 9.98E−16 |

From a dermatopathologist perspective, there is no single criterion to determine whether a pigmented lesion is a severely atypical nevus or has reached the threshold for melanoma. Dermatopathologists wrestle with at least 10 separate histologic features. No one immunohistochemical marker is able to distinguish benign from malignant melanocytic proliferations either.

The invention herein presents a melanoma biopsy assay with improved diagnostic performance in differentiating melanoma from melanocytic lesions by identifying and validating a specific genetic signature of melanoma. The testing results demonstrate a progressive increase in at least two genes that are differentially expressed in melanoma: SILV and GDF15. However, based on multivariate analysis with a linear regression model, addition of GDF15 did not improve SILV performance beyond the AUC of 0.94 in the clear-cut cases. Therefore, SILV is designated as the final marker for the melanoma biopsy assay. A significant difference was also observed between severely atypical nevi and melanoma for SILV with a p-value of 0.0077 making this marker applicable for diagnosis of both clear-cut (unequivocal) and suspicious (equivocal) cases, the latter being the most difficult challenge for expert pathologists.

TABLE 6
Sequence Descriptions, Names and SEQ ID NOs

| SEQ | | | | | |
|---|---|---|---|---|---|
| ID | Affymetrix | Gene symbol | | | |
| No. | PSID | Sequence name, | Accession No. | 5′-3′ Sequence | Gene name in NCBI |
| 1 | 209848_s_at | SILV Forward | NM_006928.3 | AGCTTATCATGCCTGGTCA | Homo sapiens silver |
| | | primer | | A | homolog (mouse) |
| 2 | | SILV Reverse | | GGGTACGGAGAAGTCTTG | (SILV), mRNA |
| | | primer | | CT | |
| 3 | | SILV Probe | | FAM- | |
| | | | | AGGTTCCGCTATCGTGGG | |
| | | | | CAT-BHQ1 | |
| 4 | 206630_at | TYR ABI AoD*, | NM_000372.4 | Hs00165976_m1 | Homo sapiens |
| | | | | | tyrosinase |
| | | | | | (oculocutaneous |
| | | | | | albinism TA) (TYR), |
| | | | | | mRNA |
| 5 | 221577_x_at | GDF15 Forward | NM_004864.1 | CGCCAGAAGTGCGGCT | Homo sapiens growth |
| | | primer | | | differentiation factor |
| 6 | | GDF15 Reverse | | CGGCCCGAGAGATACGC | 15 (GDF15), mRNA |
| | | primer | | | |
| 7 | | GDF15 MGB Probe | | FAM-ATCCGGCGGCCAC | |
| 8 | 204585_s_at | L1CAM Forward | NM_000425.2 | ACTATGGCCTTGTCTGGGA | Homo sapiens L1 cell |
| | | primer | | TCTC | adhesion molecule, |
| 9 | | L1CAM Reverse | | AGATATGGAACCTGAAGTT | mRNA |
| | | primer | | GCACTG | |
| 10 | | L1CAM MGB | | FAM- | |
| | | Probe | | CACCATCTCAGCCACAGC | |

### Full-Length Sequences of 4 Markers as Provided in NCBI Database.

>gi|113722118|ref|NM_00372.4|Homo sapiens
tyrosinase (oculocutaneous albinism TA) (TYR),
mRNA
ATCACTGTAGTAGTAGCTGGAAAGAGAAATCTGTGACTCCAATTAGCCAG
TTCCTGCAGACCTTGTGAGGACTAGAGGAAGAATGCTCCTGGCTGTTTTG
TACTGCCTGCTGTGGAGTTTCCAGACCTCCGCTGGCCATTTCCCTAGAGC
CTGTGTCTCCTCTAAGAACCTGATGGAGAAGGAATGCTGTCCACCGTGGA
GCGGGGACAGGAGTCCCTGTGGCCAGCTTTCAGGCAGAGGTTCCTGTCAG
AATATCCTTCTGTCCAATGCACCACTTGGGCCTCAATTTCCCTTCACAGG
GGTGGATGACCGGGAGTCGTGGCCTTCCGTCTTTTATAATAGGACCTGCC
AGTGCTCTGGCAACTTCATGGGATTCAACTGTGGAAACTGCAAGTTTGGC
TTTTGGGGACCAAACTGCACAGAGAGACGACTCTTGGTGAGAAGAAACAT
CTTCGATTTGAGTGCCCCAGAGAAGGACAAATTTTTTGCCTACCTCACTT
TAGCAAAGCATACCATCAGCTCAGACTATGTCATCCCCATAGGGACCTAT
GGCCAAATGAAAAATGGATCAACACCCATGTTTAACGACATCAATATTTA
TGACCTCTTTGTCTGGATGCATTATTATGTGTCAATGGATGCACTGCTTG
GGGGATCTGAAATCTGGAGAGACATTGATTTTGCCCATGAAGCACCAGCT
TTTCTGCCTTGGCATAGACTCTTCTTGTTGCGGTGGGAACAAGAAATCCA
GAAGCTGACAGGAGATGAAAACTTCACTATTCCATATTGGGACTGGCGGG
ATGCAGAAAAGTGTGACATTTGCACAGATGAGTACATGGGAGGTCAGCAC
CCCACAAATCCTAACTTACTCAGCCCAGCATCATTCTTCTCCTCTTGGCA
GATTGTCTGTAGCCGATTGGAGGAGTACAACAGCCATCAGTCTTTATGCA
ATGGAACGCCCGAGGGACCTTTACGGCGTAATCCTGGAAACCATGACAAA
TCCAGAACCCCAAGGCTCCCCTCTTCAGCTGATGTAGAATTTTGCCTGAG
TTTGACCCAATATGAATCTGGTTCCATGGATAAAGCTGCCAATTTCAGCT
TTAGAAATACACTGGAAGGATTTGCTAGTCCACTTACTGGGATAGCGGAT
GCCTCTCAAAGCAGCATGCACAATGCCTTGCACATCTATATGAATGGAAC
AATGTCCCAGGTACAGGGATCTGCCAACGATCCTATCTTCCTTCTTCACC
ATGCATTTGTTGACAGTATTTTTGAGCAGTGGCTCCGAAGGCACCGTCCT
CTTCAAGAAGTTTATCCAGAAGCCAATGCACCCATTGGACATAACCGGGA
ATCCTACATGGTTCCTTTTATACCACTGTACAGAAATGGTGATTTCTTTA
TTTCATCCAAAGATCTGGGCTATGACTATAGCTATCTACAAGATTCAGAC
CCAGACTCTTTTCAAGACTACATTAAGTCCTATTTGGAACAAGCGAGTCG
GATCTGGTCATGGCTCCTTGGGGCGGCGATGGTAGGGGCCGTCCTCACTG
CCCTGCTGGCAGGGCTTGTGAGCTTGCTGTGTCGTCACAAGAGAAAGCAG
CTTCCTGAAGAAAAGCAGCCACTCCTCATGGAGAAAGAGGATTACCACAG
CTTGTATCAGAGCCATTTATAAAAGGCTTAGGCAATAGAGTAGGGCCAAA
AAGCCTGACCTCACTCTAACTCAAAGTAATGTCCAGGTTCCCAGAGAATA
TCTGCTGGTATTTTTCTGTAAAGACCATTTGCAAAATTGTAACCTAATAC
AAAGTGTAGCCTTCTTCCAACTCAGGTAGAACACACCTGTCTTTGTCTTG
CTGTTTTCACTCAGCCCTTTTAACATTTTCCCCTAAGCCCATATGTCTAA
GGAAAGGATGCTATTTGGTAATGAGGAACTGTTATTTGTATGTGAATTAA
AGTGCTCTTATTTTAAAAAATTGAAATAATTTTGATTTTTGCCTTCTGAT
TATTTAAAGATCTATATATGTTTTATTGGCCCCTTCTTTATTTTAATAAA
ACAGTGAGAAATCTAAAAAAAAAAAAAAAAAA
>gi|153792494|ref|NM_004864.2|Homo sapiens growth
differentiation factor 15 (GDF15), mRNA
AGTCCCAGCTCAGAGCCGCAACCTGCACAGCCATGCCCGGGCAAGAACTC
AGGACGGTGAATGGCTCTCAGATGCTCCTGGTGTTGCTGGTGCTCTCGTG
GCTGCCGCATGGGGGCGCCCTGTCTCTGGCCGAGGCGAGCCGCGCAAGTT
TCCCGGGACCCTCAGAGTTGCACTCCGAAGACTCCAGATTCCGAGAGTTG
CGGAAACGCTACGAGGACCTGCTAACCAGGCTGCGGGCCAACCAGAGCTG
GGAAGATTCGAACACCGACCTCGTCCCGGCCCCTGCAGTCCGGATACTCA
CGCCAGAAGTGCGGCTGGGATCCGGCGGCCACCTGCACCTGCGTATCTCT
CGGGCCGCCCTTCCCGAGGGGCTCCCCGAGGCCTCCCGCCTTCACCGGGC
TCTGTTCCGGCTGTCCCCGACGGCGTCAAGGTCGTGGGACGTGACACGAC
CGCTGCGGCGTCAGCTCAGCCTTGCAAGACCCCAGGCGCCCGCGCTGCAC
CTGCGACTGTCGCCGCCGCCGTCGCAGTCGGACCAACTGCTGGCAGAATC
TTCGTCCGCACGGCCCCAGCTGGAGTTGCACTTGCGGCCGCAAGCCGCCA
GGGGGCGCCGCAGAGCGCGTGCGCGCAACGGGGACCACTGTCCGCTCGGG
CCCGGGCGTTGCTGCCGTCTGCACACGGTCCGCGCGTCGCTGGAAGACCT
GGGCTGGGCCGATTGGGTGCTGTCGCCACGGGAGGTGCAAGTGACCATGT
GCATCGGCGCGTGCCCGAGCCAGTTCCGGGCGGCAAACATGCACGCGCAG
ATCAAGACGAGCCTGCACCGCCTGAAGCCCGACACGGTGCCAGCGCCCTG
CTGCGTGCCCGCCAGCTACAATCCCATGGTGCTCATTCAAAAGACCGACA
CCGGGGTGTCGCTCCAGACCTATGATGACTTGTTAGCCAAAGACTGCCAC
TGCATATGAGCAGTCCTGGTCCTTCCACTGTGCACCTGCGCGGAGGACGC
GACCTCAGTTGTCCTGCCCTGTGGAATGGGCTCAAGGTTCCTGAGACACC
CGATTCCTGCCCAAACAGCTGTATTTATATAAGTCTGTTATTTATTATTA
ATTTATTGGGGTGACCTTCTTGGGGACTCGGGGGCTGGTCTGATGGAACT
GTGTATTTATTTAAAACTCTGGTGATAAAAATAAAGCTGTCTGAACTGTT
AAAAAAAAAAAAAAAAAAAA
>gi|42542384|ref|NM_006928.3|Homo sapiens silver
homolog (mouse) (SILV), mRNA
AGTGCCTTTGGTTGCTGGAGGGAAGAACACAATGGATCTGGTGCTAAAAA
GATGCCTTCTTCATTTGGCTGTGATAGGTGCTTTGCTGGCTGTGGGGGCT
ACAAAAGTACCCAGAAACCAGGACTGGCTTGGTGTCTCAAGGCAACTCAG
AACCAAAGCCTGGAACAGGCAGCTGTATCCAGAGTGGACAGAAGCCCAGA
GACTTGACTGCTGGAGAGGTGGTCAAGTGTCCCTCAAGGTCAGTAATGAT
GGGCCTACACTGATTGGTGCAAATGCCTCCTTCTCTATTGCCTTGAACTT
CCCTGGAAGCCAAAAGGTATTGCCAGATGGGCAGGTTATCTGGGTCAACA
ATACCATCATCAATGGGAGCCAGGTGTGGGGAGGACAGCCAGTGTATCCC
CAGGAAACTGACGATGCCTGCATCTTCCCTGATGGTGGACCTTGCCCATC
TGGCTCTTGGTCTCAGAAGAGAAGCTTTGTTTATGTCTGGAAGACCTGGG
GCCAATACTGGCAAGTTCTAGGGGGCCCAGTGTCTGGGCTGAGCATTGGG
ACAGGCAGGGCAATGCTGGGCACACACACCATGGAAGTGACTGTCTACCA
TCGCCGGGGATCCCGGAGCTATGTGCCTCTTGCTCATTCCAGCTCAGCCT
TCACCATTACTGACCAGGTGCCTTTCTCCGTGAGCGTGTCCCAGTTGCGG
GCCTTGGATGGAGGGAACAAGCACTTCCTGAGAAATCAGCCTCTGACCTT
TGCCCTCCAGCTCCATGACCCCAGTGGCTATCTGGCTGAAGCTGACCTCT
CCTACACCTGGGACTTTGGAGACAGTAGTGGAACCCTGATCTCTCGGGCA
CTTGTGGTCACTCATACTTACCTGGAGCCTGGCCCAGTCACTGCCCAGGT
GGTCCTGCAGGCTGCCATTCCTCTCACCTCCTGTGGCTCCTCCCCAGTTC
CAGGCACCACAGATGGGCACAGGCCAACTGCAGAGGCCCCTAACACCACA
GCTGGCCAAGTGCCTACTACAGAAGTTGTGGGTACTACACCTGGTCAGGC
GCCAACTGCAGAGCCCTCTGGAACCACATCTGTGCAGGTGCCAACCACTG
AAGTCATAAGCACTGCACCTGTGCAGATGCCAACTGCAGAGAGCACAGGT
ATGACACCTGAGAAGGTGCCAGTTTCAGAGGTCATGGGTACCACACTGGC
AGAGATGTCAACTCCAGAGGCTACAGGTATGACACCTGCAGAGGTATCAA
TTGTGGTGCTTTCTGGAACCACAGCTGCACAGGTAACAACTACAGAGTGG
GTGGAGACCACAGCTAGAGAGCTACCTATCCCTGAGCCTGAAGGTCCAGA
TGCCAGCTCAATCATGTCTACGGAAAGTATTACAGGTTCCCTGGGCCCCC
TGCTGGATGGTACAGCCACCTTAAGGCTGGTGAAGAGACAAGTCCCCCTG
GATTGTGTTCTGTATCGATATGGTTCCTTTTCCGTCACCCTGGACATTGT
CCAGGGTATTGAAAGTGCCGAGATCCTGCAGGCTGTGCCGTCCGGTGAGG
GGGATGCATTTGAGCTGACTGTGTCCTGCCAAGGCGGGCTGCCCAAGGAA
GCCTGCATGGAGATCTCATCGCCAGGGTGCCAGCCCCCTGCCCAGCGGCT
GTGCCAGCCTGTGCTACCCAGCCCAGCCTGCCAGCTGGTTCTGCACCAGA
TACTGAAGGGTGGCTCGGGGACATACTGCCTCAATGTGTCTCTGGCTGAT
ACCAACAGCCTGGCAGTGGTCAGCACCCAGCTTATCATGCCTGGTCAAGA
AGCAGGCCTTGGGCAGGTTCCGCTGATCGTGGGCATCTTGCTGGTGTTGA
TGGCTGTGGTCCTTGCATCTCTGATATATAGGCGCAGACTTATGAAGCAA
GACTTCTCCGTACCCCAGTTGCCACATAGCAGCAGTCACTGGCTGCGTCT
ACCCCGCATCTTCTGCTCTTGTCCCATTGGTGAGAATAGCCCCCTCCTCA
GTGGGCAGCAGGTCTGAGTACTCTCATATGATGCTGTGATTTTCCTGGAG
TTGACAGAAACACCTATATTTCCCCCAGTCTTCCCTGGGAGACTACTATT
AACTGAAATAAATACTCAGAGCCTGAAAAAAAAAAAAAAAAAA
>gi|13435354|ref|NM_000425.2|Homo sapiens L1 cell
adhesion molecule(L1CAM), transcript variant 1,
mRNA
GCGCGGTGCCGCCGGGAAAGATGGTCGTGGCGCTGCGGTACGTGTGGCCT
CTCCTCCTCTGCAGCCCCTGCCTGCTTATCCAGATCCCCGAGGAATATGA
AGGACACCATGTGATGGAGCCACCTGTCATCACGGAACAGTCTCCACGGC
GCCTGGTTGTCTTCCCCACAGATGACATCAGCCTCAAGTGTGAGGCCAGT
GGCAAGCCCGAAGTGCAGTTCCGCTGGACGAGGGATGGTGTCCACTTCAA
ACCCAAGGAAGAGCTGGGTGTGACCGTGTACCAGTCGCCCCACTCTGGCT
CCTTCACCATCACGGGCAACAACAGCAACTTTGCTCAGAGGTTCCAGGGC
ATCTACCGCTGCTTTGCCAGCAATAAGCTGGGCACCGCCATGTCCCATGA
GATCCGGCTCATGGCCGAGGGTGCCCCCAAGTGGCCAAAGGAGACAGTGA
AGCCCGTGGAGGTGGAGGAAGGGGAGTCAGTGGTTCTGCCTTGCAACCCT
CCCCCAAGTGCAGAGCCTCTCCGGATCTACTGGATGAACAGCAAGATCTT
GCACATCAAGCAGGACGAGCGGGTGACGATGGGCCAGAACGGCAACCTCT
ACTTTGCCAATGTGCTCACCTCCGACAACCACTCAGACTACATCTGCCAC
GCCCACTTCCCAGGCACCAGGACCATCATTCAGAAGGAACCCATTGACCT
CCGGGTCAAGGCCACCAACAGCATGATTGACAGGAAGCCGCGCCTGCTCT
TCCCCACCAACTCCAGCAGCCACCTGGTGGCCTTGCAGGGGCAGCCATTG
GTCCTGGAGTGCATCGCCGAGGGCTTTCCCACGCCCACCATCAAATGGCT
GCGCCCCAGTGGCCCCATGCCAGCCGACCGTGTCACCTACCAGAACCACA
ACAAGACCCTGCAGCTGCTGAAAGTGGGCGAGGAGGATGATGGCGAGTAC
CGCTGCCTGGCCGAGAACTCACTGGGCAGTGCCCGGCATGCGTACTATGT
CACCGTGGAGGCTGCCCCGTACTGGCTGCACAAGCCCCAGAGCCATCTAT
ATGGGCCAGGAGAGACTGCCCGCCTGGACTGCCAAGTCCAGGGCAGGCCC
CAACCAGAGGTCACCTGGAGAATCAACGGGATCCCTGTGGAGGAGCTGGC
CAAAGACCAGAAGTACCGGATTCAGCGTGGCGCCCTGATCCTGAGCAACG
TGCAGCCCAGTGACACAATGGTGACCCAATGTGAGGCCCGCAACCGGCAC
GGGCTCTTGCTGGCCAATGCCTACATCTACGTTGTCCAGCTGCCAGCCAA
GATCCTGACTGCGGACAATCAGACGTACATGGCTGTCCAGGGCAGCACTG
CCTACCTTCTGTGCAAGGCCTTCGGAGCGCCTGTGCCCAGTGTTCAGTGG
CTGGACGAGGATGGGACAACAGTGCTTCAGGACGAACGCTTCTTCCCCTA
TGCCAATGGGACCCTGGGCATTCGAGACCTCCAGGCCAATGACACCGGAC
GCTACTTCTGCCTGGCTGCCAATGACCAAAACAATGTTACCATCATGGCT
AACCTGAAGGTTAAAGATGCAACTCAGATCACTCAGGGGCCCCGCAGCAC
AATCGAGAAGAAAGGTTCCAGGGTGACCTTCACGTGCCAGGCCTCCTTTG
ACCCCTCCTTGCAGCCCAGCATCACCTGGCGTGGGGACGGTCGAGACCTC
CAGGAGCTTGGGGACAGTGACAAGTACTTCATAGAGGATGGGCGCCTGGT
CATCCACAGCCTGGACTACAGCGACCAGGGCAACTACAGCTGCGTGGCCA
GTACCGAACTGGATGTGGTGGAGAGTAGGGCACACCTCTTGGTGGTGGGG
AGCCCTGGGCCGGTGCCACGGCTGGTGCTGTCCGACCTGCACCTGCTGAC
GCAGAGCCAGGTGCGCGTGTCCTGGAGTCCTGCAGAAGACCACAATGCCC
CCATTGAGAAATATGACATTGAATTTGAGGACAAGGAAATGGCGCCTGAA
AAATGGTACAGTCTGGGCAAGGTTCCAGGGAACCAGACCTCTACCACCCT
CAAGCTGTCGCCCTATGTCCACTACACCTTTAGGGTTACTGCCATAAACA
AATATGGCCCCGGGGAGCCCAGCCCGGTCTCTGAGACTGTGGTCACACCT
GAGGCAGCCCCAGAGAAGAACCCTGTGGATGTGAAGGGGGAAGGAAATGA
GACCACCAATATGGTCATCACGTGGAAGCCGCTCCGGTGGATGGACTGGA
ACGCCCCCCAGGTTCAGTACCGCGTGCAGTGGCGCCCTCAGGGGACACGA
GGGCCCTGGCAGGAGCAGATTGTCAGCGACCCCTTCCTGGTGGTGTCCAA
CACGTCCACCTTCGTGCCCTATGAGATCAAAGTCCAGGCCGTCAACAGCC
AGGGCAAGGGACCAGAGCCCCAGGTCACTATCGGCTACTCTGGAGAGGAC
TACCCCCAGGCAATCCCTGAGCTGGAAGGCATTGAAATCCTCAACTCAAG
TGCCGTGCTGGTCAAGTGGCGGCCGGTGGACCTGGCCCAGGTCAAGGGCC
ACCTCCGCGGATACAATGTGACGTACTGGAGGGAGGGCAGTCAGAGGAAG
CACAGCAAGAGACATATCCACAAAGACCATGTGGTGGTGCCCGCCAACAC
CACCAGTGTCATCCTCAGTGGCTTGCGGCCCTATAGCTCCTACCACCTGG
AGGTGCAGGCCTTTAACGGGCGAGGATCGGGGCCCGCCAGCGAGTTCACC
TTCAGCACCCCAGAGGGAGTGCCTGGCCACCCCGAGGCGTTGCACCTGGA
GTGCCAGTCGAACACCAGCCTGCTGCTGCGCTGGCAGCCCCCACTCAGCC
ACAACGGCGTGCTCACCGGCTACGTGCTCTCCTACCACCCCCTGGATGAG
GGGGGCAAGGGGCAACTGTCCTTCAACCTTCGGGACCCCGAACTTCGGAC
ACACAACCTGACCGATCTCAGCCCCCACCTGCGGTACCGCTTCCAGCTTC
AGGCCACCACCAAAGAGGGCCCTGGTGAAGCCATCGTACGGGAAGGAGGC
ACTATGGCCTTGTCTGGGATCTCAGATTTTGGCAACATCTCAGCCACAGC
GGGTGAAAACTACAGTGTCGTCTCCTGGGTCCCCAAGGAGGGCCAGTGCA
ACTTCAGGTTCCATATCTTCTTCAAAGCCTTGGGAGAAGAGAAGGGTGGG
GCTTCCCTTTCGCCACAGTATGTCAGCTACAACCAGAGCTCCTACACGCA
GTGGGACCTGCAGCCTGACACTGACTACGAGATCCACTTGTTTAAGGAGA
GGATGTTCCGGCACCAAATGGCTGTGAAGACCAATGGCACAGGCCGCGTG
AGGCTCCCTCCTGCTGGCTTCGCCACTGAGGGCTGGTTCATCGGCTTTGT
GAGTGCCATCATCCTCCTGCTCCTCGTCCTGCTCATCCTCTGCTTCATCA
AGCGCAGCAAGGGCGGCAAATACTCAGTGAAGGATAAGGAGGACACCCAG
GTGGACTCTGAGGCCCGACCGATGAAAGATGAGACCTTCGGCGAGTACAG
GTCCCTGGAGAGTGACAACGAGGAGAAGGCCTTTGGCAGCAGCCAGCCAT
CGCTCAACGGGGACATCAAGCCCCTGGGCAGTGACGACAGCCTGGCCGAT
TATGGGGGCAGCGTGGATGTTCAGTTCAACGAGGATGGTTCGTTCATTGG
CCAGTACAGTGGCAAGAAGGAGAAGGAGGCGGCAGGGGGCAATGACACCT
CAGGGGCCACTTCCCCCATCAACCCTGCCGTGGCCCTAGAATAGTGGAGT
CCAGGACAGGAGATGCTGTGCCCCTGGCCTTGGGATCCAGGCCCCTCCCT
CTCCAGCAGGCCCATGGGAGGCTGGAGTTGGGGCAGAGGAGAACTTCCTG
CCTCGGATCCCCTTCCTACCACCCGGTCCCCACTTTATTGCCAAAACCCA
GCTGCACCCCTTCCTGGGCACACGCTGCTCTGCCCCAGCTTGGGCAGATC
TCCCACATGCCAGGGGCCTTTGGGTGCTGTTTTGCCAGCCCATTTGGGCA
GAGAGGCTGTGGTTTGGGGGAGAAGAAGTAGGGGTGGCCCGAAAGGGTCT
CCGAAATGCTGTCTTTCTTGCTCCCTGACTGGGGGCAGACATGGTGGGGT
CTCCTCAGGACCAGGGTTGGCACCTTCCCCCTCCCCCAGCCACTCCCCAG
CCAGCCTGGCTGGGACTGGGAACAGAACTCGGTGTCCCCACCATCTGCTG
TCTTTTCTTTGCCATCTCTGCTCCAACCGGGATGGGAGCCGGGCAAACTG
GCCGCGGGGGCAGGGGAGGCCATCTGGAGAGCCCAGAGTCCCCCCACTCC
CAGCATCGCACTCTGGCAGCACCGCCTCTTCCCGCCGCCCAGCCCACCCC
ATGGCCGGCTTTCAGGAGCTCCATACACACGCTGCCTTCGGTACCCACCA
CACAACATCCAAGTGGCCTCCGTCACTACCTGGCTGCGGGGCGGGCACAC
CTCCTCCCACTGCCCACTGGCCGGC

What is claimed is:

1. A method of identifying a melanoma comprising the steps of
a. obtaining a tissue sample; and
b. measuring the expression levels in the sample of genes encoding mRNA corresponding to SILV (SEQ ID No. 1-3 and 13)

2. The method of claim 1 further comprising measuring the expression level of a gene encoding tyrosinase (SEQ ID NO: 4 and 14).

3. The method of claims 1 and 2 measuring the expression level of SILV relative to TYR

4. The method of claim 1, 2, or 3 wherein the sample is a primary skin biopsy sample.

5. The method of claim 1, 2, or 3 wherein gene expression is measured on a microarray or genechip.

6. The method of claim 1, 2, or 3 wherein gene expression is determined by nucleic acid amplification conducted by polymerase chain reaction (PCR) of RNA extracted from the sample.

7. The method wherein a probability for the diagnosis of melanoma is determined from claim 1, 2 or 3.
